(19) United States
(12) Patent Application Publication
(10) Pub. No.: US 2025/0041803 A1
(43) Pub. Date: Feb. 6, 2025

(54) MOLECULAR FILTERS FOR SELECTIVE REMOVAL AND REPURPOSING OF GASEOUS FUMES AND EMISSIONS

(71) Applicant: EXPOSOME PVT. LTD., Mumbai (IN)

(72) Inventor: Prerna Goradia, Mumbai (IN)

(21) Appl. No.: 18/922,322

(22) Filed: Oct. 21, 2024

Related U.S. Application Data
(63) Continuation of application No. PCT/IN2023/050379, filed on Apr. 20, 2023.

(30) Foreign Application Priority Data
Apr. 20, 2022 (IN) 202221023163

(51) Int. Cl. B01D 53/96; A23B 7/152; A61L 9/014; A61L 101/30; B01D 53/46; B01D 53/62; B01D 53/72; B01D 53/82; B01J 20/02; B01J 20/04; B01J 20/06; B01J 20/30; B01J 20/32; B01J 23/44; B01J 37/02; B01J 37/06; B01J 37/10; G21F 9/02

(52) U.S. Cl. CPC B01D 53/96; A23B 7/152; A61L 9/014; B01D 53/46; B01D 53/62; B01D 53/72; B01D 53/82; B01J 20/0259; B01J 20/0266; B01J 20/041; B01J 20/043; B01J 20/046; B01J 20/06; B01J 20/3007; B01J 20/3071; B01J 20/3078; B01J 20/3204; B01J 20/3236; B01J 20/3293; B01J 23/44; B01J 37/0207; B01J 37/06; B01J 37/10; A61L 2101/30; A61L 2209/22; B01D 2253/1122; B01D 2253/1124; B01D 2253/25; B01D 2257/104; B01D 2257/20; B01D 2257/302; B01D 2257/304; B01D 2257/404; B01D 2257/406; B01D 2257/502; B01D 2257/60; B01D 2257/7022; B01D 2257/91; B01D 2258/06; B01D 2259/40083; G21F 9/02

(57) ABSTRACT

The present invention relates to a system and method for lowering the toxic pollutants from emissions containing molecular filter media compositions and methods of production. The filter media provides air purification by absorbing the effluents from the emission and then repurposing the contaminants through chemical interaction on the surface of the filter media and the regenerant. The adsorbent composition of the filter media maintains its integrity at high process temperature.

### RELATED APPLICATIONS

This application claims priority under 35 U.S.C. § 120 to, and is a continuation of, co-pending International Application PCT/IN2023/050379, filed Apr. 20, 2023 and designating the US, which claims priority to IN Application 202221023163, filed Apr. 20, 2022, such IN Application also being claimed priority to under 35 U.S.C. § 119. These IN and International applications are incorporated by reference herein in their entireties.

### FIELD

The present invention relates to system and method for removal of harmful compounds from toxic gases. In particular, the present invention relates to chemisorbent systems and regenerable molecular filters for abatement of effluents for emissions.

### BACKGROUND

Air pollution has become one of the most discussed issues in this era and new regulations on emissions of air pollutants obligated the companies to control their hazardous emissions. Extensive use of toxic and corrosive gases in industry has led to problems which occur upon leakage of gases in storage or flow system devices. The use of toxic and corrosive gases in the chemical and semiconductor industries has led to similar types of regulations for gas cylinders and flow systems.

Scrubbers are one of the primary devices that control gaseous emissions, especially acid gases. Dry scrubbing systems are used to get rid of pollutants and contaminants in exhaust gas without creating a wet sludge. They work by combining carefully chosen chemical reagents with the exhaust stream at incredibly high speeds which neutralizes the pollutants in the gas. The operation is done in three steps: gas cooling, reagent injection, and filtering. In the gas cooling system, emission gases are cooled to make the pollutant removal easier which is done by the use of an evaporative cooler. Once the gas has been significantly cooled the reagent injection can begin. However, the neutralized pollutants continue to remain a part of the dry scrubbing stream.

References have been made to the following literature:

US-20150182945-A1 relates to dry-scrubbing media compositions, methods of preparation and methods of use are provided. The compositions contain activated alumina and magnesium oxide. Optionally, activated carbon and other impregnates, such as hydroxides of group 1A metals, are included. The compositions exhibit improved efficiency and capacity for the removal of compounds, such as hydrogen sulfide, from an air-stream. However, once the adsorbents are spent, they have to be sent into a landfill.

US Pat. No.5,482,536-A relates to an apparatus for containing and scrubbing toxic or corrosive gases from a leaking pipe or cylinder is provided. A gas passageway attaches at one end to a leakage location on a pipe or cylinder, and at the other end to an air operated exhauster. The air operated exhauster, through the input of a non-flammable purge gas, creates an exhaust flow from the leakage location to a drum which is attached to the air operated exhauster. The drum contains a scrubbing media which, when it contacts the leaked gas, cleans or removes the harmful component, thus allowing release to the atmosphere of the cleaned air.

WO-2005025733 relates to A dry scrubbing system for treatment of effluent from an upstream effluent-generating process. The dry scrubbing system accommodates operation in a process window involving substantial variation in process conditions, e.g., flow rate and/or concentration, of scrubbable gas species in the effluent. Multiple scrubbing media are utilized in the dry scrubbing system, each optimal in a regime of the operating window, and together ensuring at least a predetermined level of removal of scrubbable gas species over the entire operating window.

US-20180169578-A1 relates to a specific aspect, gaseous phosphorus in the effluent is abated by use of potassium hydroxide as an active abatement agent. The invention relates to a process for removing mercury from flue gases from combustion plants, wherein the process comprises providing an adsorbent based on carbon, producing an aqueous suspension comprising the adsorbent, introducing the suspension into the flue gas stream from the combustion plants into the dry gas phase of the flue gas which is undersaturated with water vapor and loading the adsorbent with mercury over a predetermined reaction path, keeping the mercury-laden adsorbent out of the flue gas stream and landfilling or regenerating the mercury-laden adsorbent.

It is evident that despite the widespread use of activated carbon and zeolites for the treatment of emissions, they have a limited durability. None of the compositions described to date have effectively solved problems surrounding the efficacy and regenerability of dry scrubbers. To counteract these claims, a reusable and robust platform and materials to reduce pollutants and provide clean air in the atmosphere is the need of the hour. The molecular filter media, of the present invention, are extremely high surface area filter powders and pellets for lowering concentration of toxic pollutants and is a cost-effective technology that performs for difficult-to-remove pollutants. Plus, the technology focuses on circular economy of using pollutants as a feedstock for the next step. For example, hydrogen sulphide gas can be repurposed into other sulphide salts.

The information disclosed in this background of the disclosure section is only for enhancement of understanding of the general background of the invention and should not be taken as an acknowledgement or any form of suggestion that this information forms the prior art already known to a person skilled in the art.

### SUMMARY

In a principal object of the present invention, molecular filter compositions, methods of preparing same, and system for removing the contaminants from the emissions are provided.

Another object of the present invention is to provide molecular filter compositions with improved efficiency and capacity for the removal of toxic gaseous compounds from the areas such as petroleum refineries and storage areas, sewage treatment facilities, hospital morgues, animal rooms, and pulp and paper factories.

The present invention attempts to overcome the problems faced in the prior art, and discloses a system and regenerable molecular media for removal of toxic pollutants from the emissions and a preparation method thereof.

The compositions contain activated inert media components containing high surface areas and are often impregnated with reactants that effectively trap and neutralize the gaseous pollutants. Optionally, activated carbon and other impregnates, are included in the compositions. The compositions exhibit improved efficiency and capacity for the removal of toxic gaseous compounds from the areas such as petroleum refineries and storage areas, sewage treatment facilities, hospital morgues, animal rooms, and pulp and paper factories.

In an embodiment of the present invention, the invention discloses a method for removing contaminants from gas emissions comprising providing gas emissions in a reaction chamber. In an embodiment providing a dry or semi-dry molecular filter media in the reaction chamber for trapping and neutralizing the gaseous pollutants from the gas emissions to generate cleaned gas, wherein the filter media comprises 30-85% by weight activated substrates and 15-25% by weight active reagents and providing at least one gas outlet for the release of the cleaned gas. Upon saturation of active sites of the filter media by the pollutants, the filter media is recharged by flushing the reaction chamber with a regenerant comprising at least one or more liquid, gas or solid particles dispersed in a liquid. This is followed by recirculating the regenerant after recharging the filter media and reusing the filter media for cleaning the toxic gas emissions to render it safe for environmental release and in process repurposing the contaminant into by products.

In another embodiment of the present invention, the activated substrate is at least one of carbon, metal oxides, metal carbides, ceramics, porcelain, clay, porous glass, mixed metal oxides comprising binary, ternary and quaternary with respect to the presence of the number of different metal cations and combinations thereof but not limited to. In an embodiment, the metal oxides can function both as solid catalysts, either as active phases or as supports. Metal oxides are utilized both for their acid—base and redox properties.

In a preferrable embodiment of the present invention, the active reagent is at least one of reducers like metabisulphite, permanganates, oxidizers, organic acids, salts of trisodium citrate and other acids and bases, metals salts of iron, silver, zinc, palladium and combinations thereof, but not limited to. In an embodiment, the regenerant is aqueous solution with the actives comprising one or more solid particles, liquid or gases such as air, oxygen, hydrogen, ozone dispersed in a liquid.

In an exemplary embodiment of the present invention, the invention discloses a rechargeable filter media composition for removing toxic components from gas emissions comprising 30-85% by weight activated substrates and 15-25% by weight active reagents. In an embodiment, the actives are active ingredients for facilitating the reactions of adsorption of the toxic components and neutralization thereof.

In an embodiment of the present invention, the invention discloses a process for making molecular filter media comprising the steps of mixing activated beads in water for at least 10 minutes, followed by decanting the water and washing/rinsing the activated beads with distilled water to remove impurities; and incubating the washed and activated beads for at least one hour at 60° C. temperature, to obtain high-surface area activated substrate; to obtain active reagent, taking at least 5% wt/vol of actives and optionally mixing it with at least 10-20% wt/vol of metal oxide powder followed by adding distilled water into the mixture as per needed and drying the mixture; finally impregnating high-surface area activated substrate with active reagent in a ratio 1:1 to 5:1; extruding and spheronizing the mixture into pellets to make the molecular filter media.

In another embodiment of the present invention, the drying is by at least one of air drying, heat drying and blow drying. In an embodiment, the filter media is formed as a high-surface area carbon or high-surface area metal oxide pellet which is impregnated with active reagents which adsorbs the pollutants in the emissions.

In an exemplary embodiment of the present invention, the invention discloses a system for removing toxic components from gas emissions comprising at least one gas inlet in a reaction chamber with a gas passageway for drawing gas emissions into the reaction chamber. In an embodiment, the said reaction chamber is packed with a molecular filter media for trapping and neutralizing the toxic components from the gas emissions to generate cleaned gas and providing at least one gas outlet for release of the cleaned gas into the environment. Further, providing at least an inlet for adding a regenerant into the reaction chamber for recharging the filter media and removing the regenerant from at least an outlet at one end of the reaction chamber, where the regenerant repurposes the contaminant for reuse.

In yet another embodiment of the present invention, the filter media removes the contaminants by at least one of trapping and neutralizing, catalysts and redox transformation. In another embodiment, the regenerant repurposes the contaminant species into salts for use as raw material in industry by at least one of thermal evaporation, membrane technology, osmosis, reverse osmosis and combinations thereof but not limited to.

In an exemplary embodiment of the present invention, the invention discloses a regenerant for recharging the filter media comprising aqueous solution with the actives and or gases such as air, oxygen, hydrogen and or ozone. In an embodiment the active is one or more liquid, gas or solid particles dispersed in a liquid.

In another embodiment, the active component in the regenerant is at least one or more liquid, gas or solid particles such as NaOH, oxidants, potassium bicarbonate, Palladium and combinations thereof but not limited to. In an embodiment, the active is recharged into the filter media composition by at least one mechanism selected from neutralization, catalysis or redox transformation.

In an embodiment of the present invention, the invention discloses an efficient, inexpensive method for filtering harmful, toxic or odorous compounds from an air or gas stream.

In another embodiment of the present invention, the invention discloses an adsorbent composition that maintains its integrity at high process temperature.

According to this invention, there is provided a process for making molecular filter media comprising:
mixing, 30-85% by final weight, activated beads in about twice quantity of water for at least 10 minutes;
decanting the water and washing/rinsing the activated beads with distilled water to remove impurities;
incubating the washed and activated beads for at least one hour at 60° C. temperature in order to obtain a high-surface area activated substrate;
taking at least 5% wt/vol of actives and, optionally, mixing it with at least 10-20% wt/vol of metal oxide powder;
adding distilled water into the mixture (of step (d));
drying the mixture (of step (e)) in order to obtain an active reagent wherein the active reagents are active ingredients for facilitating the reactions of adsorption of the toxic components and neutralization thereof;
impregnating the obtained high-surface area activated substrate (of step (c)) with the obtained activated reagent (of step (f)) in a ratio 1:1 to 5:1 in order to obtain an impregnated mixture; and
extruding and spheronizing the impregnated mixture (of step g) into pellets to make the molecular filter media.

According to this invention, there is also provided a method for removing contaminants from gas emissions comprising:
providing gas emissions in a reaction chamber;
providing a dry or semi-dry molecular filter media in the reaction chamber for trapping and neutralizing the gaseous pollutants from the gas emissions to generate cleaned gas, wherein the filter media comprises 30-85% by weight activated substrates and 15-25% by weight active reagents;
providing at least one gas outlet for the release of the cleaned gas;
upon saturation of active sites of the filter media by the pollutants, recharging the same by flushing the reaction chamber with a regenerant comprising at least one or more liquid, gas or solid particles dispersed in a liquid; and
recirculating the regenerant after recharging the filter media and reusing the filter media for cleaning the toxic gas emissions to render it safe for environmental release and in process repurposing the contaminant into by products.

According to this invention, there is also provided a rechargeable filter media composition for removing toxic components from gas emissions comprising:
30-85% by weight activated substrates;
15-25% by weight active reagents, wherein the actives are active ingredients for facilitating the reactions of adsorption of the toxic components and neutralization thereof.

According to this invention, there is also provided a system for removing toxic components from gas emissions comprising:
at least one gas inlet in a reaction chamber with a gas passageway for drawing gas emissions into the reaction chamber, wherein the said reaction chamber is packed with a molecular filter media for trapping and neutralizing the toxic components from the gas emissions to generate cleaned gas;
providing at least one gas outlet for release of the cleaned gas into the environment;
providing at least an inlet for adding a regenerant into the reaction chamber for recharging the filter media and removing the regenerant from at least an outlet at one end of the reaction chamber,
wherein the regenerant repurposes the contaminant for reuse;
wherein the filter media removes the contaminants by at least one of trapping and neutralizing, catalysts and redox transformation; and
the regenerant repurposes the contaminant species into salts for use as raw material in industry by at least one of thermal evaporation, membrane technology, osmosis, reverse osmosis and combinations thereof but not limited to.

In at least an embodiment, drying is by at least one of air drying, heat drying and blow drying.

In at least an embodiment, said regenerant is a water-based formulation which has about 4 to 10 percent actives that wash away the by-product of the gas and molecular filter and infuse the active back into the molecular filter readying it for the next cycle.

In at least an embodiment, said regenerant comprises aqueous solutions with the actives and or gases such as air, oxygen, hydrogen and or ozone. one or more liquid, gas or solid particles dispersed.

In at least an embodiment, said active reagent is at least one of reducers like metabisulphite, permanganates, oxidizers, organic acids salts of trisodium citrate and other acids and bases, metals salts of iron, silver, zinc, palladium and combinations thereof.

In at least an embodiment, an active component in the regenerant is at least one or more liquid, gas or solid particles such as NaOH, oxidants, potassium bicarbonate, palladium and combinations thereof.

In at least an embodiment, said activated substrate is at least one of carbon, metal oxides, metal carbides, ceramics, porcelain, clay, porous glass, mixed metal oxides comprising binary, ternary and quaternary with respect to the presence of the number of different metal cations and combinations thereof.

In at least an embodiment, said filter media is formed as a high-surface area carbon or high-surface area metal oxide pellet which is impregnated with active reagents which adsorbs the pollutants in the emissions.

In at least an embodiment, said filter media are palladium coated substrates for removing carbon monoxide from the emissions and converting into CO2 as a by-product, wherein the by product is trapped by the regenerant NaOH and converted into the carbonate salts, yielding a repurposed by-product for the industry by use of catalysis and neutralization reactions.

In at least an embodiment, said filter media is an oxidizer with permanganate coated substrates for removing ethylene from rotting vegetables and converting into carbon dioxide as a by-product, wherein the by-product forms a blanket on the produce and the regenerant is an oxidant for replenishing the media.

In at least an embodiment, wherein said filter media is metabisulphite coated substrates for removing oxygen from produce.

These and other objects, features and advantages of the present invention will become apparent after a review of the following detailed description of the disclosed embodiments and the appended claims.

### DETAILED DESCRIPTION

Because this is a patent document, general broad rules of construction should be applied when reading it. Everything described and shown in this document is an example of subject matter falling within the scope of the claims, appended below. Any specific structural and functional details disclosed herein are merely for purposes of describing how to make and use examples. Several different embodiments and methods not specifically disclosed herein may fall within the claim scope; as such, the claims may be embodied in many alternate forms and should not be construed as limited to only examples set forth herein.

The terms “comprises”, “comprising”, or any other variations thereof used in the disclosure, are intended to cover a non-exclusive inclusion, such that a device, apparatus, system, assembly, method that comprises a list of components or a series of steps that does not include only those components or steps but may include other components or steps not expressly listed or inherent to such apparatus, or assembly, or device. In other words, one or more elements or steps in a system or device or process proceeded by “comprises . . . a” or “comprising . . . of” does not, without more constraints, preclude the existence of other elements or additional elements or additional steps in the system or device or process as the case may be.

Rather, exclusive modifiers like “only” or “singular” may preclude presence or addition of other subject matter in modified terms. The use of permissive terms like “may” or “can” reflect optionality such that modified terms are not necessarily present, but absence of permissive terms does not reflect compulsion. In listing items in example embodiments, conjunctions and inclusive terms like “and,” “with,” and “or” include all combinations of one or more of the listed items without exclusion. The use of “etc.” is defined as “et cetera” and indicates the inclusion of all other elements belonging to the same group of the preceding items, in any “and/or” combination(s). Modifiers “first,” “second,” “another,” etc. may be used herein to describe various items, but they do not confine modified items to any order. These terms are used only to distinguish one element from another; where there are “second” or higher ordinals, there merely must be that many number of elements, without necessarily any difference or other relationship among those elements.

When an element is related, such as by being “connected,” “coupled,” “on,” “attached,” “fixed,” etc., to another element, it can be directly connected to the other element, or intervening elements may be present. In contrast, when an element is referred to as being “directly connected,” “directly coupled,” etc. to another element, there are no intervening elements present. Other words used to describe the relationship between elements should be interpreted in a like fashion (e.g., “between” versus “directly between,” “adjacent” versus “directly adjacent,” etc.).

As used herein, singular forms like “a,” “an,” and “the” are intended to include both the singular and plural forms, unless the language explicitly indicates otherwise. Indefinite articles like “a” and “an” introduce or refer to any modified term, both previously-introduced and not, while definite articles like “the” refer to the same previously-introduced term. Relative terms such as “almost” or “more” and terms of degree such as “approximately” or “substantially” reflect 10% variance in modified values or, where understood by the skilled artisan in the technological context, the full range of imprecision that still achieves functionality of modified terms. Precision and non-variance are expressed by contrary terms like “exactly.”

The inventor has recognized that despite the widespread use of the available treatments for lowering pollutants, it is not adequately lowered and operational expenditures are also very high. Further, due to filtering media being used up during the process, operational costs are high. Thus, a need remains for a novel and inventive regenerant filter media methodology in such applications which besides environmentally friendly is both economical as well as provide excellent results for removing the pollutants. To overcome these issues, the inventor has developed example embodiments and methods described below to address these and other problems recognized by the inventors with unique solutions enabled by example embodiments.

In an embodiment, the present invention relates to a system and method for lowering the toxic pollutants from emissions containing molecular filter media compositions and methods of production. The filter media provides air purification and odor control by absorbing and adsorbing odors and then destroying the collected odors through chemical interaction on the surface of the filter media.

The system comprises a regenerable molecular filter filled in a packed bed configuration. The corrosive gas is made to flow through it where the pollutant is selectively trapped in the molecular media and is in turn filtered. As, the active sites of the filter media get saturated with the pollutants/contaminants from the emission, the regenerant is made to flow in which removes the trapped contaminants and replenishes the actives in the molecular filter. The regenerant repurposes and converts the pollutants into salts which can be used as a byproduct for other industry. In an embodiment, a filter for removing CO2 works with NaOH solution in the regenerant and the byproduct of the reaction is sodium carbonate.

In an embodiment, the molecular media composition described herein contains activated alumina and metal carbonate. In one embodiment, the molecular filter media further includes powdered activated carbon. In another embodiment, the molecular filter media is impregnated with reducing agents such as metabisulphite.

In an embodiment, the molecular filter media compositions provided herein have an enhanced capacity for the adsorption of certain undesired compounds at a higher efficiency than currently available media. In addition, the molecular filter media compositions described herein enjoy increased physical strength. In an embodiment, the extremely high surface area carbons are used. Activated carbon, powdered carbon and carbon black are collectively referred to herein as “high surface area carbon.” The size range of the powder is largely a matter of choice, although when producing pellets of filter media, some parameters are necessary for ensuring that uniform pellets are achieved during rolling. In one embodiment, carbon is sized for passing 85% through a 40 by 80 mesh screens. It has been noted that the rate of adsorption can vary upon the surface area of the activated carbon used. Accordingly, it is important to employ activated carbons having high surface areas, preferably between 500 and 2000 m2/g surface area, and most preferably between 1000-1200 m2/g.

In an embodiment of the present invention, the invention discloses a method for removing contaminants from gas emissions comprising providing gas emissions in a reaction chamber. In an embodiment containing a dry or semi-dry molecular filter media in the reaction chamber for trapping and neutralizing the gaseous pollutants from the gas emissions to generate cleaned gas, wherein the filter media comprises 30-85% by weight activated substrates and 15-25% by weight active reagents and providing at least one gas outlet for the release of the cleaned gas. Upon saturation of active sites of the filter media by the pollutants, the filter media is recharged by flushing the reaction chamber with a regenerant comprising at least one or more liquid, gas or solid particles dispersed in a liquid. This is followed by recirculating the regenerant after recharging the filter media and reusing the filter media for cleaning the toxic gas emissions to render it safe for environmental release and in process repurposing the contaminant into by products.

In another embodiment of the present invention, the activated substrate is at least one of carbon, metal oxides, metal carbides, ceramics, porcelain, clay, porous glass, mixed metal oxides comprising binary, ternary and quaternary with respect to the presence of the number of different metal cations and combinations thereof but not limited to. In an embodiment, the metal oxides can function both as solid catalysts, either as active phases or as supports. Metal oxides are utilized both for their acid—base and redox properties. In a preferrable embodiment of the present invention, the active reagent is at least one of reducers like metabisulphite, permanganates, oxidizers, organic acids salts of trisodium citrate and other acids and bases, metals salts of iron, silver, zinc, palladium and combinations thereof, but not limited to. In an embodiment, the regenerant is aqueous solution with the actives comprising one or more solid particles, liquid or gases such as air, oxygen, hydrogen, ozone dispersed in a liquid.

In an exemplary embodiment of the present invention, the invention discloses a rechargeable filter media composition for removing toxic components from gas emissions comprising 30-85% by weight activated substrates and 15-25% by weight active reagents. In an embodiment, the actives are active ingredients for facilitating the reactions of adsorption of the toxic components and neutralization thereof.

In another embodiment of the present invention, the activated substrate is at least one of carbon, metal oxides, metal carbides, ceramics, porcelain, clay, porous glass, mixed metal oxides comprising binary, ternary and quaternary with respect to the presence of the number of different metal cations and combinations thereof but not limited to. In an embodiment, the active reagent is at least one of reducers like metabisulphite, permanganates, oxidizers, organic acids salts of trisodium citrate and other acids and bases, metals salts of iron, silver, zinc, palladium and combinations thereof, but not limited to.

In an embodiment of the present invention, the invention discloses a process for making molecular filter media comprising the steps of mixing activated beads in water for at least 10 minutes, followed by decanting the water and washing/rinsing the activated beads with distilled water to remove impurities; and incubating the washed and activated beads for at least one hour at 60° C. temperature, to obtain high-surface area activated substrate; to obtain active reagent, taking at least 5% wt/vol of actives and optionally mixing it with at least 10-20% wt/vol of metal oxide powder followed by adding distilled water into the mixture as per needed and drying the mixture; finally impregnating high-surface area activated substrate with active reagent in a ratio 1:1 to 5:1; extruding and spheronizing the mixture into pellets to make the molecular filter media.

In another embodiment of the present invention, the drying is by at least one of air drying, heat drying and blow drying. In an embodiment, the filter media is formed as a high-surface area carbon or high-surface area metal oxide pellet which is impregnated with active reagents which adsorbs the pollutants in the emissions.

In an exemplary embodiment of the present invention, the invention discloses a system for removing toxic components from gas emissions comprising at least one gas inlet in a reaction chamber with a gas passageway for drawing gas emissions into the reaction chamber. In an embodiment, the reaction chamber is packed with a molecular filter media for trapping and neutralizing the toxic components from the gas emissions to generate cleaned gas and providing at least one gas outlet for release of the cleaned gas into the environment. Further, providing at least an inlet for adding a regenerant into the reaction chamber for recharging the filter media and removing the regenerant from at least an outlet at one end of the reaction chamber, where the regenerant repurposes the contaminant for reuse.

In yet another embodiment of the present invention, the filter media removes the contaminants by at least one of trapping and neutralizing, catalysts and redox transformation. In another embodiment, the regenerant repurposes the contaminant species into salts for use as raw material in industry by at least one of thermal evaporation, membrane technology, osmosis, reverse osmosis and combinations thereof but not limited to.

In still another embodiment of the present invention, the filter media is palladium coated substrates for removing carbon monoxide from the emissions and converting into CO2 as a by-product, wherein the by product is trapped by the regenerant NaOH and converted into the carbonate salts, yielding a repurposed by-product for the industry by use of catalysis and neutralization reactions.

In a preferred embodiment, the filter media is an oxidizer such as permanganate coated substrates for removing ethylene from rotting vegetables and converting into carbon dioxide as a by-product, wherein the by product also forms a blanket on the produce and the regenerant used is oxidant for replenishing the media. In another embodiment, the filter media is metabisulphite coated substrates for removing oxygen from produce.

In an exemplary embodiment of the present invention, the invention discloses a regenerant for recharging the filter media comprising aqueous solution with the actives and or gases such as air, oxygen, hydrogen and or ozone. In an embodiment the active is one or more liquid, gas or solid particles dispersed in a liquid.

In another embodiment, the active component in the regenerant is at least one or more liquid, gas or solid particles such as NaOH, oxidants, potassium bicarbonate, Palladium and combinations thereof but not limited to. In an embodiment, the active is recharged into the filter media composition by at least one mechanism selected from neutralization, catalysis or redox transformation.

The molecular filter media compositions provided herein utilize a chemisorptive process that removes chlorine and sulfur dioxide by means of absorption, adsorption and chemical reaction. Chlorine or sulfur dioxide are trapped within the filter media where an irreversible chemical reaction takes place, changing the gas into harmless solids and salts which are then concentrated in the regenerant. In an embodiment, the filter media involves contacting an effluent gas stream with a solid material which functions to chemisorb or react with the undesired components to affect their removal.

In another embodiment, the molecular filter media described herein concentrates and fully contains hazardous contaminants, is passive in operation, has no moving parts and works on demand, making it a safe and preferable mode of filtration and purification. The regenerant for the molecular filter is wet or dry (ozone, hydrogen gas, others).

In another embodiment, the molecular filter media is prepared or manufactured by extrusion to form a solid structure. Extrusion is a manufacturing process used to create long objects of a fixed cross-sectional profile. A material, or mixture, is pushed and/or drawn through a die of the desired profile shape. The die may be of different shapes and diameters. Plasticity and shape retention of extruded materials can be varied or improved through the use of binders. The binder is not particularly limited and there may be used any of various types of binders which is used when a molded product is made by extrusion molding or injection molding. More specifically, there are exemplified various types of alcohols, celluloses such as methyl cellulose, ethyl cellulose etc., starches, vinyl resins, various waxes, thermoplastic polyolefins such as polyethylene, polypropylene etc., polyacetate vinyl compounds etc. In one embodiment, binders include but are not limited to, starch adhesives, organic binders, clay, and feldspar. Undesirable airborne compounds, including chlorine and sulfur containing compounds, ammonia, formaldehyde, urea, carbon monoxide, oxides of nitrogen, mercaptans, amines, and ethylene, occur in a number of environments, where most primarily are responsible for the presence of disagreeable odors, irritating or toxic gases.

In another embodiment the system is used for other application such as food preservation. For food preservation the removal of oxygen by reducing agents such as sodium metabisulphite helps in food preservation and slowing of metabolism of the microbes. Other materials that can be considered as reductants are ferrous carbonate and ascorbic acid. The reducing agent can be stored in an external tank and introduced into the filters to “recharge” the media so that a continuous process occurs. Oxidizing agents such as permanganate also help to break down the ethylene which is the “rotting” gas in food and blanket the food with the product gas which delays the rotting process.

Selective removal of sulfide is also important and apart from the chemical removal
Ozonation can be used to oxidize the matter along with the media. The active passage of ozone and other reactive gases along with the media can be considered to yield industrially relevant materials. The thought is that the gaseous waste is a misplaced resource. Additionally, it can also be absorbed in liquid or gel type of media. Example Sulfur dioxide (SO2) which is a colorless gas can be oxidized to sulfur trioxide using ozone—which, which in the presence of water is readily transformed to sulphuric acid which will have industrial uses. Health effects caused by exposure to high levels of SO2 include breathing problems, respiratory illness, changes in lung defenses, worsening respiratory and cardiovascular disease. Therefore, the removal of the sulphur dioxide is extremely important.

Removal of radioactivity also becomes important in nuclear establishments and mixture of potassium hydroxide and potassium iodides can help for this purpose. Our formulations also involve the mixture of sodium hydroxide and magnesium chloride. As clean energy from nuclear reactors becomes important the safety around the process would involve the deployment of absorbent media around the reactors in case any leakage is detected.

In an embodiment, the system relates to the removal of pollutants such as controlling emissions of Hydrogen sulfide (H2S)gas particularly from municipal sewage treatment plants; Ammonia (NH3), produced in animal rooms and nurseries, formaldehyde (HCHO) present in hospital morgues;urea (CH4N2O) present in toilet exhaust and paper industry to soften cellulose; Carbon monoxide (CO); Oxides of nitrogen, including nitrogen dioxide (NO2) nitric oxide (NO), and nitrous oxide (N2O ); Mercaptans and amines, including methyl mercaptan (CH3SH), butyl mercaptan (C4H9SH) and methyl amine (CH5N), and other undesirable gases present in sewerage odor. Ozonation coupled with smart materials media can help in the oxidation of ethylene. Our absorbents containing either oxidizing agents or reducing agents such as sodium metabisulphite are also important.

In an embodiment, the regenerable molecular filter media of the present invention are a high total adsorption capacity for the targeted compound, high efficiency in removing the compound from an air or gas stream, and a high ignition temperature (non-flammability). This absorbent composition attempts to absorb numerous undesirable gaseous compounds that are present in household air.

### Example 1: Media for Removal of SOx, NO_{X}

The filter media was produced as per the scheme containing: for activated substrate, the alumina and zeolite powder; and active reagent by mixing at least 6% wt/vol of actives such as KMnO4 with at least 10-20 wt/vol of zeolite powder in another beaker and adding distilled water into the mixture as per needed, followed by drying the composition by air drying/heat drying/blow drying and this is active reagent; impregnating substrate with active in a ratio 1:1 to 5:1 and heated to 350 C for extruding, spheronizing into pellets. Further the regenerant for this SOx, NOX lowering filter media was bleach, peroxide, ozone gas, oxygen gas.

### Example 2: Media for Removal of Acidic Gases Containing Chlorine

The filter media was produced as per the scheme containing: activated substrate is the coarse activated carbon and zeolite powder; active reagent, mixing at least 4% wt/vol of actives such as NaOH with at least 10-20% wt/vol of zeolite powder in another beaker and adding distilled water into the mixture as per needed, followed by drying the composition by air drying/heat drying/blow drying; impregnating substrate with active in a ratio 1:1 to 5:1 and heated to 350° C. for extruding, spheronizing into pellets. Further the regenerant for these acidic gases lowering filter media was 4% NaOH in water.

### Example 3: Media for Removal of Ammonia

The filter media was produced as per the scheme containing: activated substrate here is the coarse activated carbon and zeolite powder; for active reagent, mixing at least 4%-85% wt/vol of actives such as phosphoric acid with at least 10-20% wt/vol of zeolite powder in another beaker and adding distilled water into the mixture as per needed, followed by drying the composition by air drying/heat drying/blow drying and this is active reagent; impregnating substrate with active in a ratio 1:1 to 5:1 and heated to 350° C. for extruding, spheronizing into pellets. Further the regenerant for these acidic gases lowering filter media was 4%-85% phosphoric acid in water.

### Example 4: Media for Removal of Radioactive Materials

The filter media was produced as per the scheme containing: activated substrate here is the coarse activated carbon and zeolite powder; for active reagent, mixing actives such as at least 4% wt/vol KOH and 4% wt/vol of KI with at least 10-20% wt/vol of zeolite powder in another beaker and adding distilled water into the mixture as per needed, followed by drying the composition by air drying/heat drying/blow drying and this is active reagent; impregnating substrate with active in a ratio 1:1 to 5:1 and heated to 350° C. for extruding, spheronizing into pellets. Further the regenerant for these acidic gases lowering filter media was KOH and KI in water.

### Example 5: Media for Removal of Heavy Metals From Air and Water Such as As and Pb

The filter media was produced as per the scheme containing: activated substrate here is the iron oxide ceramic and coarse activated carbon (30%+70% ratio); for active reagent, mixing at least 4% wt/vol of actives such as iron hydroxide reagent with at least 10-20% wt/vol of zeolite powder in another beaker and adding distilled water into the mixture as per needed, followed by drying the composition by air drying/heat drying/blow drying and this is active reagent; impregnating substrate with active in a ratio 1:1 to 5:1 and heated to 350° C. for extruding, spheronizing into pellets. Further the regenerant for these acidic gases lowering filter media was finely dispersed iron powder/dissolved iron salt in water and if required compressed gas flows.

### Example 6: Media for Removal of Bacteria and Virus

The filter media was produced as per the scheme containing: activated substrate here is the coarse activated carbon and zeolite powder; for active reagent, mixing at least 4% wt/vol of actives such as 3 metals mixture selected from silver, zinc and copper with at least 10-20% wt/vol of zeolite powder in another beaker and adding distilled water into the mixture as per needed, followed by drying the composition by air drying/heat drying/blow drying and this is active reagent; impregnating substrate with active in a ratio 1:1 to 5:1 and heated to 350° C. for extruding, spheronizing into pellets. Further the regenerant for these acidic gases lowering filter media was an aqueous formulation with silver, zinc and copper in water.

### Example 7: Media for Removal of H₂S 20% by Weight, SOx 10% by Weight, Chlorine 10% by weight

The filter media was produced as per the scheme containing: activated substrate here is the alumina balls and zeolite powder; for active reagent, mixing at least 32% wt/vol of actives such as potassium bicarbonate with at least 10-20% wt/vol of zeolite powder in another beaker and adding distilled water into the mixture as per needed, followed by drying the composition by air drying/heat drying/blow drying and this is active reagent; impregnating substrate with active in a ratio 1:1 to 5:1 and heated to 350° C. for extruding, spheronizing into pellets. Further the regenerant for these acidic gases lowering filter media was potassium bicarbonate in water. The experiment resulted in the adsorption of a total 20% of H2S.

### Example 8: Media for Removal of NOx, Carbon Monoxide and Hydrocarbons

The filter media was produced as per the scheme containing: activated substrate is the coarse activated carbon and metal oxide powder; for active reagent, mixing at least 0.1% wt/vol of actives such as palladium with at least 10-20% wt/vol of zeolite powder in another beaker and adding distilled water into the mixture as per needed, followed by drying the composition by air drying/heat drying/blow drying and this is active reagent; impregnating substrate with active in a ratio 1:1 to 5:1 and heated to 350° C. for extruding, spheronizing into pellets. Further the regenerant for these acidic gases lowering filter media was palladium activator in water.

### Example 9: Media for Removal of Fish Oil

The filter media was produced as per the scheme containing: activated is the coarse activated carbon and metal oxide powder; for active reagent, mixing at least 5 to 10% wt/vol of actives such as trisodium citrate with at least 10-20% wt/vol of ceramic powder in another beaker and adding distilled water into the mixture as per needed, followed by drying the composition by air drying/heat drying/blow drying and this is active reagent; impregnating substrate with active in a ratio 1:1 to 5:1 and heated to 350° C. for extruding, spheronizing into pellets. Further the regenerant for these acidic gases lowering filter media was aqueous solutions with the actives and or gases such as air, oxygen, hydrogen and or ozone.

### Example 10: Media for Removal of Ethylene and Controlling the Quality of Fruits and Vegetables Produce

For this the filter media was oxidizer such as permanganate coated substrates for removing ethylene from rotting vegetables and in turn was converting into carbon dioxide as a by-product, wherein the by product also formed a blanket on the produce and the regenerant used was oxidant for replenishing the media. This was done for removing exogenous food rotting gases from the atmosphere, gas accumulation and its harmful effect on vegetables during post-harvest storage as its important for both nutritional and organoleptic quality of vegetables. Measurements with fruit's pH, the degradation of ascorbic acid during storage time and antioxidant capacity proved the efficacy of the described media.

TECHNICAL ADVANTAGES: Filter media is landfill disposable and does not require specialist disposal and costs associated therewith; The media is also non-toxic and non-hazardous before and after reaction or usage; The filter media operates effectively at room temperatures; The filter media, while having enhanced chlorine and sulfur dioxide removal capacity over media known and currently used in the art, gives the benefit of wet-scrubbing systems with the filter media resulting in enhanced corrosive gas removal; The regenerant gets enfused with important salts and value-added products are formed which are used in the same or different industrial processes; Compared to wet scrubbers where the efficiency of trapping of the corrosive gas is very low and there is a small quantity of the salt mixed in large (over 90%) water here the salt content is very high which leads to efficient recovery of the salts; The molecular filter media is an adsorbent composition that maintains its integrity at high process temperature.

It is to be understood that one of ordinary skill in the art may develop or modify the filter media described herein in addition to the pelletization and extrusion methods discussed above. Indeed, it is envisaged that the filter media may have any suitable size, shape and conformation appropriate to the end use application and the removal of contaminants, such as chlorine and sulfur dioxide from gas or air-streams. The filter media may further include active ingredients and inactive ingredients, and may be in a finely divided form, e.g., beads, spheres, rings, toroidal shapes, irregular shapes, rods, cylinders, flakes, films, cubes, polygonal geometric shapes, sheets, coils, helices, meshes, granules, pellets, powders, particulates, extrudates, honeycomb matrix, composites (of the filter media with other components), or crushed forms of the foregoing conformations.

It will be further appreciated that functions or structures of a plurality of components or steps may be combined into a single component or step, or the functions or structures of one-step or component may be split among plural steps or components. The present invention contemplates all of these combinations. Unless stated otherwise, dimensions and geometries of the various structures depicted herein are not intended to be restrictive of the invention, and other dimensions or geometries are possible. In addition, while a feature of the present invention may have been described in the context of only one of the illustrated embodiments, such feature may be combined with one or more other features of other embodiments, for any given application. It will also be appreciated from the above that the fabrication of the unique structures herein and the operation thereof also constitute methods in accordance with the present invention. The present invention also encompasses intermediate and end products resulting from the practice of the methods herein. The use of “comprising” or “including” also contemplates embodiments that “consist essentially of” or “consist of” the recited feature.

Although embodiments for the present invention have been described in language specific to structural features, it is to be understood that the present invention is not necessarily limited to the specific features described. Rather, the specific features and methods are disclosed as embodiments for the present invention. Numerous modifications and adaptations of the system/component of the present invention will be apparent to those skilled in the art, and thus it is intended by the appended claims to cover all such modifications and adaptations which fall within the scope of the present invention.

1. A method of making molecular filter media, the method comprising:
mixing 30-85% by final weight activated beads in about twice quantity of water for at least 10 minutes;
decanting the water and washing and/or rinsing the activated beads with distilled water;
incubating the washed and activated beads for at least one hour at 60° C. temperature to obtain a high surface area activated substrate;
impregnating the high surface area activated substrate with an activated reagent in a ratio of about 1-5:1 to obtain an impregnated mixture, wherein the active reagents adsorb and neutralize toxic components; and
extruding and spheronizing the impregnated mixture into pellets to make the molecular filter media.

2. The method of claim 1, further comprising:
introducing a regenerant into the filter media to wash away by-products of the filter media and infuse active reagents back into the filter media readying it for a next cycle, wherein the regenerant is a water-based formulation which has about 4 to 10 percent active reagents.

3. The method of claim 2, wherein an active component in the regenerant is liquid, gas, or solid particles of at least one of NaOH, oxidants, potassium bicarbonate, and palladium.

4. The method of claim 2, wherein the regenerant includes the active reagents and at least one of oxygen, hydrogen, ozone, and a liquid, gas, or solid particulate.

5. The method of claim 2, wherein the filter media is a palladium-coated substrate configured to convert carbon monoxide into CO2, and wherein the regenerant is NaOH configured to convert the CO2 into carbonate salts.

6. The method of claim 2, wherein the filter media is an oxidizer with a permanganate-coated substrate configured to convert ethylene into carbon dioxide that forms a blanket on produce, and wherein the regenerant is an oxidant configured to regenerate the filter media.

7. The method of claim 1, wherein the active reagent is at least one of a reducer, permanganate, oxidizer, organic acids salt, metals salts of iron, silver, zinc, and palladium.

8. The method of claim 1, wherein the activated substrate is at least one of, carbon, metal oxides, metal carbides, ceramics, porcelain, clay, porous glass, and mixed metal oxides of binary, ternary and quaternary with respect to the presence of the number of different metal cations.

9. The method of claim 1, wherein the filter media is formed as a high-surface area carbon or high-surface area metal oxide pellet impregnated with active reagent that adsorbs the pollutants in the emissions.

10. The method of claim 1, wherein the filter media is metabisulphite coated substrates configured to remove oxygen from produce.

11. The method of claim 1, further comprising:
mixing distilled water, 10-20% wt/vol of metal oxide powder, and at least 5% wt/vol of active components to obtain a reagent mixture, and
drying the reagent mixture to obtain the activated reagent.

12. A rechargeable filter media composition for removing toxic components from gas emissions, the media comprising:
30-85% by weight activated substrates; and
15-25% by weight active reagents that facilitate adsorption and neutralization of toxic components.

13. The media of claim 12, further comprising:
a regenerant configured to wash away by-products of the filter media and infuse active reagents back into the filter media readying it for a next cycle, wherein the regenerant is a water-based formulation which has about 4 to 10 percent active reagents.

14. A system for removing toxic components from gas emissions comprising:
a gas inlet in a reaction chamber with a gas passageway for drawing gas emissions into the reaction chamber, wherein the reaction chamber is packed with a molecular filter media for trapping and neutralizing toxic components from the gas emissions to generate cleaned gas;
a gas outlet for release of the cleaned gas into the environment;
an inlet for adding a regenerant into the reaction chamber for recharging the filter media; and
an outlet at one end of the reaction chamber, wherein the regenerant repurposes the contaminant for reuse, wherein the filter media removes the toxic components by at least one of trapping and neutralizing, catalysts, and redox transformation, and wherein the regenerant repurposes the contaminant species into salts for use as raw material in industry by at least one of thermal evaporation, membrane technology, osmosis, and reverse osmosis.

15. The system of claim 14, wherein the regenerant is configured to wash away by-products of the filter media and infuse active reagents back into the filter media readying it for a next cycle, wherein the regenerant is a water-based formulation which has about 4 to 10 percent active reagents.
